(19) United States
(12) Patent Application Publication
(10) Pub. No.: US 2008/0315155 A1
(43) Pub. Date: Dec. 25, 2008

(54) Diamines Having Reduced Color

(75) Inventors: John Y. Lee, Baton Rouge, LA (US); David W. Owens, Baton Rouge, LA (US); Richard D. Glass, Baton Rouge, LA (US); Paul L. Wiggins, Baton Rouge, LA (US)

Correspondence Address: ALBEMARLE CORPORATION, 451 FLORIDA STREET, BATON ROUGE, LA, 70801-1765, US

(73) Assignee: ALBEMARLE CORPORATION, Baton Rouge, LA (US)

(21) Appl. No.: 12/096,133

(22) PCT Filed: Dec. 21, 2006
(86) PCT No.: PCT/US2006/062501

§ 371 Date: Jun. 4, 2008

Related U.S. Application Data
(60) Provisional application No. 60/755,736, filed on Dec. 30, 2005.

(51) Int. Cl. C07C 209/26; C09K 3/00

(52) U.S. Cl. 252/182.23; 564/305; 564/396

(57) ABSTRACT

This invention provides compositions which comprise at least one aromatic secondary diamine having a Gardner color number no more than about 6. The aromatic secondary diamine either is in the form of one benzene ring having two secondary amino groups on the ring, or is in the form of two benzene rings connected by an alkylene bridge and having one secondary amino group on each ring. At least one N,N′-dihydrocarbylhydroxylamine is optionally present in the composition. Processes for producing such compositions are also provided.

### TECHNICAL FIELD

This invention relates to aromatic secondary diamines having reduced coloration.

### BACKGROUND

Aromatic secondary diamines are indicated to be useful as chain extenders in the preparation of polyurethane, polyurea, and polyurethane-urea polymers and/or as curing agents for epoxy resins. In addition, at least some aromatic secondary diamines have reactivities in a desired range, and also impart satisfactory properties in the products made by their use. For certain applications, reduced color is preferable or necessary. It would be useful to have aromatic secondary diamines having reduced color in addition to both a suitable reactivity and an impartation of satisfactory properties in the products produced therefrom.

### SUMMARY OF INVENTION

This invention provides aromatic secondary diamines having reduced color, and processes for preparing aromatic secondary diamines having reduced color. The reduction in color allows the use of these diamines in applications involving lenses and glass where clarity and transparency are important. Advantageously, the color of aromatic secondary diamines can be reduced using mild conditions, and/or available reagents.

An embodiment of this invention is a composition which comprises at least one aromatic secondary diamine having a Gardner color number no more than about 6. The aromatic secondary diamine either is in the form of one benzene ring having two secondary amino groups on the ring, or is in the form of two benzene rings connected by an alkylene bridge and having one secondary amino group on each ring. At least one N,N-dihydrocarbylhydroxylamine is optionally present in the composition.

Another embodiment of this invention is a process for reducing color in an aromatic secondary diamine. The process comprises heating, while under a vacuum, at least one aromatic secondary diamine, optionally in the presence of at least one N,N-dihydrocarbylhydroxylamine. The aromatic secondary diamine either is in the form of one benzene ring having two secondary amino groups on the ring, or is in the form of two benzene rings connected by an alkylene bridge and having one secondary amino group on each ring.

Still another embodiment of this invention is a process for forming an aromatic secondary diamine which comprises mixing together a ketone or aldehyde and an aromatic primary diamine, characterized in that the process is conducted in the substantial absence of oxygen, and optionally in the presence of at least one N,N-dihydrocarbylhydroxylamine. The aromatic primary diamine is either is in the form of one benzene ring having two primary amino groups on the ring, or is in the form of two benzene rings connected by an alkylene bridge and having one primary amino group on each ring.

These and other embodiments and features of this invention will be still further apparent from the ensuing description and appended claims.

### FURTHER DETAILED DESCRIPTION OF THE INVENTION

Some terms that are commonly used in the art can be used to refer to certain aspects of the present invention. In particular, the formation of a secondary amine from a primary amine and an aldehyde or ketone is often referred to as reductive alkylation or reductive amination, and the terms “reductive alkylation” and “reductive amination” can be used to describe some of the processes of the invention.

Throughout this document, the term “color-minimizing amount” generally means a quantity sufficient to reduce the existing coloration of a color-possessing aromatic secondary diamine by a measurable amount, provided the resultant reduced coloration is, by measurement, less than the coloration produced by adding to another sample of the same color-possessing aromatic secondary diamine an equal amount of a clear, colorless inert organic diluent soluble in such diamine.

Those of skill in the art will recognize that there are several ways to name the aromatic secondary diamines in this invention. For example, the structure

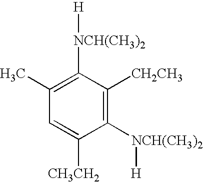

can be called N,N′-di-isopropyl-2,4-diethyl-6-methyl-1,3-benzenediamine, N,N′-di-isopropyl-2,4-diethyl-6-methyl-1,3-phenylenediamine, N,N′-di-isopropyl-3,5-diethyl-2,4-diaminotoluene, or N,N′-di-isopropyl-3,5-diethyl-toluene-2,4-diamine. Similarly, the structure

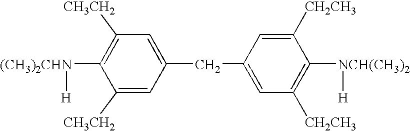

can be called N,N′-di-isopropyl-4,4′-methylenebis(2,6-diethylbenzeneamine), N,N′-di-isopropyl-4,4′-methylenebis(2,6-diethylaniline), or N,N′-di-isopropyl-3,3′,5,5′-tetraethyl-4,4′-diaminodiphenylmethane.

### Compositions of the Invention

The compositions of the invention have a Gardner color number no more than about 6. Preferably, the compositions have a Gardner color number no more than about 5; more preferred compositions have a Gardner color number no more than about 4. Even more preferred are compositions having a Gardner color number no more than about 3.5. As is known in the art, the lower the Gardner color, the clearer (less colored) the liquid appears.

Gardner color is a well known standard color measurement for liquids, and is typically applied to liquids having a yellow, reddish, and/or brownish coloration. See in this connection ASTM standards D1544 (visual standard) and D6166 (instrumentation standard).

A composition of the invention comprising one or more additives in addition to the aromatic secondary amine (e.g., a N,N-dihydrocarbylhydroxylamine and/or an optical brightener) can be formed by mixing together the aromatic secondary amine and such other additive(s) in the desired proportions.

Compositions having two or more aromatic secondary diamines are within the scope of this invention. Where an amount is stated to be used or present relative to the aromatic secondary diamine when two or more such diamines are present, that amount is understood to be relative to the combined total of the aromatic secondary diamines (e.g., relative to their combined total weight), unless otherwise stated.

A. Aromatic Secondary Diamines

The aromatic secondary diamines in the compositions of the invention are either in the form of one benzene ring having two secondary amino groups on the ring, or are in the form of two benzene rings connected by an alkylene bridge and having one secondary amino group on each ring.

Throughout this document, the term “amino hydrocarbyl group” refers to the hydrocarbyl group bound to a nitrogen atom of the aromatic secondary diamine which hydrocarbyl group is not the benzene ring to which the nitrogen atom is bound in order to form the aromatic secondary diamine. The alkylene bridge of the two-benzene-ring secondary diamine has from one to about six carbon atoms; preferably, the alkylene bridge has from one to about three carbon atoms. More preferably, the alkylene bridge has one or two carbon atoms; highly preferred is an alkylene bridge having one carbon atom, i.e., a methylene group.

The amino hydrocarbyl groups of the aromatic secondary diamine generally have from two to about twenty carbon atoms; the amino hydrocarbyl group may be aliphatic (straight chain, branched, or cyclic) or aromatic. Preferably, the amino hydrocarbyl groups are straight chain or branched chain alkyl groups having from three to about six carbon atoms. Examples of suitable amino hydrocarbyl groups include ethyl, propyl, isopropyl, 1-cyclopropylethyl, n-butyl, sec-butyl, cyclobutyl, 2-ethylbutyl, 3,3-dimethyl-2-butyl, 3-pentyl, 3-penten-2-yl, cyclopentyl, 2-(4-methylpentyl), 2,5-dimethylcyclopentyl, 2-cyclopentenyl, hexyl, cyclohexyl, methylcyclohexyl, menthyl, ionyl, phoryl, isophoryl, 2-heptyl, 4-heptyl, 2,6,-dimethyl-3-heptyl, cyclooctyl, 5-nonyl, decyl, 10-undecenyl, dodecyl, benzyl, 2,4-dimethylbenzyl, 2-phenylethyl, 1-phenylpentyl, 1-naphthyl, 2-naphthyl, 1-naphthylethyl, and the like. Particularly preferred amino hydrocarbyl groups are isopropyl and sec-butyl.

Aromatic secondary diamines with two secondary amino groups on one benzene ring preferably have the secondary amino groups meta relative to each other. In such preferred aromatic secondary diamines, the amino hydrocarbyl group preferably is a straight chain or branched chain alkyl group having from three to about six carbon atoms.

Preferred aromatic secondary diamines in which one secondary amino group is on each of two benzene rings, where the two benzene rings are connected via an alkylene bridge, have both secondary amino groups para relative to the alkylene bridge. A particularly preferred aromatic secondary diamine is a compound in which the alkylene bridge is a methylene group; this is especially preferred when the amino hydrocarbyl groups are isopropyl or sec-butyl groups.

One type of aromatic secondary diamine in the compositions of the invention is that in which at least one position ortho (immediately adjacent) to each secondary amino group has a hydrogen atom as a substituent. This type of aromatic secondary diamine is preferred; more preferred are aromatic secondary diamines in which both positions ortho to the amino group have hydrogen atoms as substituents. Examples of this type of aromatic secondary diamine include, but are not limited to, N,N′-diisopropyl-1,2-benzenediamine, N,N′-di-sec-butyl-1,3-benzenediamine, N,N′-di(2-butenyl)-1,4-benzenediamine, N,N′-dicyclopentyl-(4-ethyl-1,2-benzenediamine), N,N′-di-sec-butyl-(4-tert-butyl-1,3-benzenediamine), N,N′-di(1-cyclopropylethyl)-2-pentyl-1,4-benzenediamine, N,N′-di(4-hexyl)-(4-methyl-5-heptyl-1,3-benzenediamine), N,N′-dicyclopentyl-4,6-di-n-propyl-1,3-benzenediamine, N,N′-di-sec-butyl-(2,3-diethyl-1,4-benzenediamine), N,N′-di(1-penten-3-yl)-4,5,6-trihexyl-1,3-benzenediamine, N,N′-di(3-hexyl)-2,2′-methylenebis(benzeneamine), N,N′-di(2-cyclopentenyl)-2,3′-methylenebis(benzeneamine), N,N′-diisopropyl-2,4′-methylenebis(benzeneamine), N,N′-di-sec-butyl-3,3′-methylenebis(benzeneamine), N,N′-di(3-methyl-2-cyclohexenyl)-3,4′-methylenebis(benzeneamine), N,N′-di(3,3-dimethyl-2-butyl)-4,4′-methylenebis-(benzeneamine), N,N′-di-sec-butyl-4,4′-methylenebis(benzeneamine), N,N′-di(10-undecenyl)-4,4′-(1,2-ethanediyl)bisbenzeneamine, N,N′-di(phoryl)-3,4′-(1,3-propanediyl)bis-(benzeneamine), N,N′-di(2,4-dimethyl-3-pentyl)-2,2′-methylenebis(5-tert-butylbenzeneamine), N,N′-di(2,5-dimethylcyclopentyl)-3,3′-methylenebis(2-methylbenzeneamine), N,N′-di(isophoryl)-3,3′-methylenebis(5-pentylbenzeneamine), N,N′-di(2-hexyl)-3,3′-methylenebis(6-isopropylbenzeneamine), N,N′-dicyclohexyl-4,4′-methylenebis(3-sec-butylbenzeneamine), N,N′-di(1-cyclopentylethyl)-4,4′-(1,2-ethanediyl)bis(2-methylbenzeneamine), N,N′-diisopropyl-3,3′-methylenebis(2,4-dipentylbenzeneamine), N,N′-di-sec-butyl-3,3′-methylenebis(5,6-diisopropylbenzeneamine), and N,N′-di(menthyl)-4,4′-methylenebis(2,3-di-sec-butylbenzeneamine). An especially preferred aromatic secondary diamine of this type is N,N′-di-sec-butyl-4,4′-methylenebis(benzeneamine).

Another type of aromatic secondary diamine in the compositions of the invention is that in which each position ortho to a secondary amino group (—NHR) bears a hydrocarbyl group. The hydrocarbyl groups ortho to the secondary amino groups on the benzene rings may be the same or different. Examples of suitable hydrocarbyl groups on the benzene ring include methyl, ethyl, propyl, isopropyl, n-butyl, sec-butyl, t-butyl, pentyl, cyclopentyl, hexyl, cyclohexyl, methylcyclohexyl, heptyl, octyl, cyclooctyl, nonyl, decyl, dodecyl, phenyl, benzyl, and the like. When an aromatic secondary diamine of this type is in the form of two benzene rings connected by an alkylene bridge and having one secondary amino group on each ring and the secondary amino group is adjacent (ortho) to the alkylene bridge, the alkylene bridge is considered as a hydrocarbyl group ortho to the secondary amino group. Preferred hydrocarbyl groups on the benzene rings (ortho to a secondary amino group) of the aromatic secondary diamines are straight chain or branched chain alkyl groups having from one to about six carbon atoms; particularly preferred hydrocarbyl groups are methyl, ethyl, isopropyl, butyl, and mixtures of two or more of these groups. Here, the preference for butyl groups includes n-butyl, sec-butyl, and t-butyl groups.

Aromatic secondary diamines in the compositions of this invention having both secondary amino groups on one benzene ring and in which each position ortho (immediately adjacent) to a secondary amino group (—NHR) bears a hydrocarbyl group include, but are not limited to, N,N′-diisopropyl-2,4,6-triethyl-1,3-benzenediamine, N,N′-di-sec-butyl-2,4,6-triethyl-1,3-benzenediamine, N,N′-di-2-pentyl-2,4,6-triethyl-1,3-benzenediamine, N,N′-diisopropyl-(2,4-diethyl-6-methyl-1,3-benzenediamine), N,N′-di-sec-butyl-(2,4-diethyl-6-methyl-1,3-benzenediamine), N,N′-diisopropyl-(4,6-diethyl-2-methyl-1,3-benzenediamine), N,N′-di-sec-butyl-(4,6-diethyl-2-methyl-1,3-benzenediamine), N,N′-di(2-naphthyl)-(4,6-diethyl-2-methyl-1,3-benzenediamine), N,N′-di(2-cyclopentenyl)-(2,4-diisopropyl-6-methyl-1,3-benzenediamine), N,N′-diisopropyl-(2-methyl-4,6-di-sec-butyl-1,3-benzenediamine), N,N′-di-sec-butyl-(2-methyl-4,6-di-sec-butyl-1,3-benzenediamine), N,N′-di(1-cyclopropylethyl)-(2-methyl-4,6-di-sec-butyl-1,3-benzenediamine), N,N′-di(3,3-dimethyl-2-butyl)-(2-ethyl-4-isopropyl-6-methyl-1,3-benzenediamine), N,N′-diisopropyl-2,4,5,6-tetra-n-propyl-1,3-benzenediamine, N,N′-di(3-penten-2-yl)-2,4,5,6-tetra-n-propyl-1,3-benzenediamine, and N,N′-di(4-hexyl)-2,3,5,6-tetraethyl-1,4-benzenediamine. Particularly preferred aromatic diamines having both amino groups on one benzene ring are N,N′-diisopropyl-(2,4-diethyl-6-methyl-1,3-benzenediamine), N,N′-diisopropyl-(4,6-diethyl-2-methyl-1,3-benzenediamine), and mixtures thereof; N,N′-di-sec-butyl-(2,4-diethyl-6-methyl-1,3-benzenediamine), N,N′-di-sec-butyl-(4,6-diethyl-2-methyl-1,3-benzenediamine), and mixtures thereof.

Examples of aromatic secondary diamines of the invention in which one secondary amino group is on each of two benzene rings and in which each position ortho (immediately adjacent) to a secondary amino group (—NHR) bears a hydrocarbyl group include N,N′-diisopropyl-2,2′-methylenebis(6-n-propylbenzeneamine), N,N′-di-sec-butyl-2,2′-methylenebis(3,6-di-n-propylbenzene amine), N,N′-di(2,4-dimethylbenzyl)-2,2′-methylenebis(5,6-dihexylbenzeneamine), N,N′-diisopropyl-3,3′-methylenebis(2,6-di-n-butylbenzeneamine), N,N′-di(2,4-dimethyl-3-pentyl)-3,3′-methylenebis(2,6-di-n-butylbenzeneamine), N,N′-diisopropyl-4,4′-methylenebis(2,6-diethylbenzeneamine), N,N′-di-sec-butyl-4,4′-methylenebis(2,6-diethylbenzeneamine), N,N′-di(2-hexyl)-4,4′-methylenebis(2,6-diethylbenzeneamine), N,N′-di(1-naphthylethyl)-4,4′-methylenebis(2,6-diisopropylbenzeneamine), N,N′-dicyclobutyl-4,4′-methylenebis(2-isopropyl-6-methylbenzeneamine), N,N′-di(1-penten-3-yl)-4,4′-methylenebis(2-methyl-6-tert-butylbenzeneamine), N,N′-di-sec-butyl-4,4′-(1,2-ethanediyl)bis(2,6-diethylbenzeneamine), N,N′-di(1-cyclopentylethyl)-4,4′-(1,2-ethanediyl)bis(2,6-diethylbenzeneamine), N,N′-di(2-ethylbutyl)-4,4′-(1,2-ethanediyl)bis(2,6-diisopropylbenzeneamine), N,N′-di(10-undecenyl)-2,2′-methylenebis(3,4,6-tripentylbenzeneamine), N,N′-di(4-heptyl)-3,3′-methylenebis(2,5,6-trihexylbenzeneamine), N,N′-dimenthyl-4,4′-methylenebis(2,3,6-trimethylbenzeneamine), N,N′-dibenzyl-4,4′-methylenebis(2,3,4,6-tetramethylbenzeneamine), and the like. Particularly preferred aromatic diamines in which one amino group is on each of two benzene rings are N,N′-diisopropyl-4,4′-methylenebis(2,6-diethylbenzeneamine) and N,N′-di-sec-butyl-4,4′-methylenebis(2,6-diethylbenzeneamine).

B. N,N-Dihydrocarbylhydroxylamines

The N,N-dihydrocarbylhydroxylamines used in the practice of this invention have two hydrocarbyl groups and a hydroxyl group bound to the nitrogen atom. Each hydrocarbyl group is independently a hydrocarbyl group having up to about twenty carbon atoms; preferably, each hydrocarbyl group has up to about 8 carbon atoms. The hydrocarbyl groups of the N,N-dihydrocarbylhydroxylamine may be, for example, alkyl groups (straight chain, branched, or cyclic), alkenyl groups, cycloalkyl groups, cycloalkenyl groups, aryl groups, or aralkyl groups. In the practice of this invention, two or more N,N-dihydrocarbylhydroxylamines can be used.

Also within the scope of this invention is the use of a N,N-dihydrocarbylhydroxylamine in which the two hydrocarbyl groups taken together constitute a single divalent hydrocarbyl group bonded to the nitrogen atom such that the nitrogen atom is part of a heterocyclic ring; such single divalent hydrocarbyl group typically contains up to about 20 carbon atoms and preferably up to about 10 carbon atoms. Since the single divalent hydrocarbyl group has two different carbon atoms singly bonded to the nitrogen atom, such single divalent group may be viewed as two individual hydrocarbyl groups joined together. Thus, throughout this document the term “N,N-dihydrocarbylhydroxylamine” includes such single divalent hydrocarbyl groups that form a hetrocyclic ring containing the nitrogen atom as the heteroatom.

Suitable N,N-dialkylhydroxylamines for the practice of this invention include, but are not limited to, N,N-dipropylhydroxylamine, N,N-diisopropylhydroxylamine, N,N-dibutylhydroxylamine, N,N-diisobutylhydroxylamine, N,N-dipentylhydroxylamine, N,N-dicyclopentylhydroxylamine, N,N-di(2-cyclopentenyl)hydroxylamine, N,N-dihexylhydroxylamine, N,N-diheptylhydroxylamine, N,N-di(methylcyclohexyl)hydroxylamine, N,N-di(4-methylpentyl)hydroxylamine, N,N-di(dodecyl)hydroxylamine, N,N-di(pentadecyl)hydroxylamine, N,N-di(octadecyl)hydroxylamine, N,N-diphenylhydroxylamine, N,N-di(1-naphthyl)hydroxylamine, N,N-di(2-naphthyl)hydroxylamine, N,N-dibenzylhydroxylamine, N,N-di(4-methylbenzyl)hydroxylamine, N,N-bis(2,4-dimethylbenzyl)hydroxylamine, N,N-di(2-phenethyl)hydroxylamine, N,N-di(1-naphthylmethyl)hydroxylamine, and N,N-di(2-naphthylmethyl)hydroxylamine. Considerations when choosing a N,N-dihydrocarbylhydroxylamine include that it not evaporate or decompose during preparation or processing of the composition of which the N,N-dihydrocarbylhydroxylamine is part, and that the N,N-dihydrocarbylhydroxylamine not adversely affect the properties of the composition.

Preferred N,N-dihydrocarbylhydroxylamines are N,N-dialkylhydroxylamines; more preferred are those in which the alkyl groups are straight chain or branched chain alkyl groups, especially those in which each alkyl group, independently, has from one to about six carbon atoms. N,N-diaralkylhydroxylamines are also preferred N,N-dihydrocarbylhydroxylamines, especially those in which each aralkyl group, independently, has from seven to about fourteen carbon atoms. Particularly preferred N,N-dihydrocarbylhydroxylamines in the practice of this invention are N,N-diethylhydroxylamine and N,N-dibenzylhydroxylamine.

The amount of N,N-dihydrocarbylhydroxylamine, when present in a composition of the invention, is normally present in a color-minimizing amount. Preferably, the N,N-dihydrocarbylhydroxylamine is in the range of about 0.01 wt % to about 1.5 wt % relative to the aromatic secondary diamine. More preferably, in the range of about 0.5 wt % to about 0.75 wt % N,N-dihydrocarbylhydroxylamine relative to the aromatic secondary diamine is used. Deviations from these preferred ranges are within the scope of this invention, as amounts other than those in the preferred ranges occasionally may be needed to have a color-minimizing effect. An amount greater than a color-minimizing amount can be added when desired, for example for storage of the aromatic secondary diamine(s).

C. Optical Brighteners

One particular type of additive that is useful as part of a composition of the invention is an optical brightener. It has been found that the use of optical brighteners, especially those that can function as blue dyes, minimize the color of aromatic secondary diamines, either used alone or in combination with a N,N-dihydrocarbylhydroxylamine. Two or more optical brighteners can be present in the compositions of this invention. A particularly preferred optical brightener in the practice of this invention is a mixture of Solvent Violet 13 and Solvent Green 3 (Exalite® Blue 78-13, Exiton Inc., Dayton, Ohio).

Generally, the optical brightener is present in a color-minimizing amount. An amount of optical brightener greater than a color-minimizing amount can be used, if desired. Whether used alone or in combination with a N,N-dihydrocarbylhydroxylamine, the amount of optical brightener is preferably in the range of about optical brightener More preferably, the amount of optical brightener is generally in the range of about 1 ppm to about 10 ppm relative to the aromatic secondary diamine. While the use of larger amounts of optical brighteners is possible, it has been found that there appears to be an upper limit after which further color minimization is not achieved by the addition of more optical brightener, often because the light transmission and/or clarity of the composition, when used in certain applications, becomes too low.

A convenient way to include an optical brightener in a composition of the invention is by the use of a solution of the optical brightener in a polyol. Typically, the optical brightener is made into a solution in the polyol, which solution is then combined with the other components of the composition. Usually, the optical brightener is in the range of about 0.01 wt % to about 10 wt % in the polyol solution; preferably, the optical brightener is in the range of about 0.03 wt % to about 3 wt % in the polyol solution. The suitability of a particular polyol may depend on the end use of the composition. Preferred polyols that can be used include polyether polyols (e.g., Voranol® polyols, Dow Chemical Co.); linear polycaprolactone polyols (e.g., Tone™ polyols, Dow Chemical Co.); and amine-terminated polyols (e.g., Jeffamine® polyols, Huntsman Chemical).

As mentioned above for the N,N-dihydrocarbylhydroxylamines, considerations when choosing an optical brightener or other additive(s) (below) include that the optical brightener and/or other additive(s) not evaporate or decompose during processing of the composition of which such optical brightener(s) and/or other additive(s) is part, and that the optical brightener(s) and/or other additive(s) not adversely affect the properties of the composition.

D. Other Additives

Other additives may be added to the composition to impart desired properties to the composition, or to an end product made therefrom. One or more such additives may be made part of a composition of the invention. The additive(s) should be chosen so that the desirable properties of the composition are not adversely affected. Examples of such additives include stabilizers, including heat stabilizers and light stabilizers, ultraviolet absorbers, fluorescent agents, antifogging agents, weather-proofing agents, antistatic agents, lubricants, surfactants, antioxidants, viscosity reducing agents, dispersants, release agents, processing aids, nucleating agents, and plasticizers. The additive(s) must be compatible with the aromatic secondary diamine, and must not materially interfere with the color-minimizing activity of the N,N-dihydrocarbylhydroxylamine when present in the composition.

E. Conditions

It is recommended and preferred that the compositions of this invention be kept under an inert atmosphere to minimize the amount of oxygen present with the composition. The inert atmosphere is usually comprised of one or more inert gases, such as, for example, nitrogen, helium, or argon. Alternatively, the compositions of this invention can be kept under a vacuum, although this is often not practical. Without wishing to be bound by theory, it is believed that oxygen reacts with a portion of the composition to form an N-oxide impurity and/or the degradation product of such N-oxide impurity, which are thought to be the source of at least a portion of the coloration observed in many aromatic secondary diamines.

### Processes of the Invention

The following processes of the invention can be employed to produce compositions of the invention, i.e., aromatic secondary diamines having Gardner color numbers no more than about 6.

A. Process for Reducing Color in an Aromatic Secondary Diamine

One way to reduce the color in an aromatic secondary diamine is by heating, while under a vacuum, an aromatic secondary diamine, optionally in the presence of at least one N,N-dihydrocarbylhydroxylamine. By reducing the color of an aromatic secondary diamine, this process is able to produce a composition of the invention. However, on a cautionary note, it has been found that when the aromatic secondary diamine has been aged in the presence of oxygen or heated prior to establishment of a vacuum, the procedure of heating the aromatic secondary diamine while under a vacuum does not seem effective to reduce the color present in the aromatic secondary diamine.

The aromatic secondary diamines used in this process are those described above in the compositions of this invention. Preferred aromatic secondary diamines are also as detailed above. The N,N-dihydrocarbylhydroxylamines optionally present during the process, as well as the preferred N,N-dihydrocarbylhydroxylamines are as described above for the compositions of the invention. Amounts of the N,N-dihydrocarbylhydroxylamines in this process are preferably in the range of about 0.5 wt % to about 5 wt % relative to the aromatic secondary diamine. More preferably, in the range of about 0.5 wt % to about 2 wt % N,N-dihydrocarbylhydroxylamine relative to the aromatic secondary diamine is used. As above, deviations from these preferred ranges are within the scope of this invention, as amounts other than those in the preferred ranges may be needed to have a color-minimizing effect.

The process is conducted by placing the aromatic secondary diamine under vacuum and heating the aromatic secondary diamine while maintaining the vacuum. The vacuum is usually on the order of millitorr, preferably about 1 to about 10 millitorr, more preferably, about 1 to about 5 millitorr. Higher vacuum is possible but not necessary to obtain the reduction in color achieved in the practice of this invention. The aromatic secondary diamine is generally heated to a temperature in the range of about 30° C. to about 140° C.; more preferably, the temperature is in the range of about 45 to about 130° C. Without wishing to be bound by theory, it is thought that the heating under vacuum decomposes at least a portion of the colored impurity or impurities present with the aromatic secondary diamine. Thus, it is recommended and preferred that any further processing of the aromatic secondary diamine be conducted in the substantial absence of oxygen.

B. Process for Forming an Aromatic Secondary Diamine

Another process of the invention is a process for forming an aromatic secondary diamine which comprises mixing together a ketone or aldehyde and an aromatic primary diamine. The process is characterized in that it is conducted in the substantial absence of oxygen, and optionally in the presence of at least one N,N-dihydrocarbylhydroxylamine.

As stated above for the compositions, without wishing to be bound by theory, in the processes of this invention, the presence of oxygen is generally thought to cause formation of colored by-products. Thus, the substantial absence of oxygen during the process is believed to minimize the color of the produced aromatic secondary diamine.

The term “substantial absence of oxygen” means that oxygen is generally not present during the process. However, adventitious amounts of oxygen (e.g., at parts per million levels), although undesired, may be present during the process. It is to be understood that the presence of such adventitious amounts of oxygen are encompassed by the term “substantial absence of oxygen.”

It is recommended and preferred that the compositions of this invention be kept under a non-oxygen atmosphere to maintain a substantial absence of oxygen during the process. When the process for producing an aromatic secondary diamine uses hydrogen gas, the hydrogen alone may be the non-oxygen atmosphere. While the use of hydrogen alone is preferable, the hydrogen may be present in the process in combination with an inert gas such as nitrogen, helium, or argon (the inert gas in this situation is sometimes called a carrier gas). When the process does not employ hydrogen gas, an inert atmosphere is preferably present to assist in the exclusion of oxygen during the process. The inert atmosphere is usually comprised of one or more inert gases, such as, for example, nitrogen, helium, or argon.

The N,N-dihydrocarbylhydroxylamines used in this process and the preferences therefor are as described above for the compositions of the invention. The amount of N,N-dihydrocarbylhydroxylamine is as described above for the process for reducing color in an aromatic secondary diamine.

There are several methods for making an aromatic secondary diamine from an aromatic primary diamine and a ketone or aldehyde. Several such methods are detailed in U.S. Application No. 60/665,915, filed Mar. 28, 2005. An especially preferred method involves the use of hydrogen gas and a hydrogenation catalyst, especially where the hydrogenation catalyst is sulfided platinum on carbon, sulfided palladium on carbon, or a mixture thereof.

The aromatic primary diamines used in this process of the invention are either in the form of one benzene ring having two secondary amino groups on the ring, or are in the form of two benzene rings connected by an alkylene bridge and having one secondary amino group on each ring.

Ketones and aldehydes used in this process are hydrocarbyl ketones and hydrocarbyl aldehydes. The hydrocarbyl portion of the ketone or aldehyde may be aliphatic (cyclic, branched, or straight chain), unsaturated, aromatic, or alkylaromatic. The hydrocarbyl portion is preferably aliphatic, alkylaromatic, or aromatic. More preferably, the hydrocarbyl portion of the aldehyde or ketone is an aliphatic straight chain or a branched aliphatic group. Preferably, the ketones and aldehydes used in the practice of this invention have from three to about twenty carbon atoms. More preferred are ketones and aldehydes having from three to about fifteen carbon atoms.

A hydrogenation agent (including hydrogen gas and a hydrogenation catalyst) is used in the process. Other reagents that can be employed in the process for forming an aromatic secondary diamine include one or more of: acid ion exchange resins, solvents, and/or water removal agents.

If desired, the above process entailing heating while under vacuum may be performed on an aromatic secondary diamine formed by this process of the invention. Performing the above process of heating while under vacuum on an aromatic secondary diamine formed in this process is a preferred way to operate. Whether or not the process of heating while under vacuum is performed on the aromatic secondary amine, it is recommended and preferred that further processing of the aromatic secondary diamine, including isolation from the reaction mixture, be conducted in the substantial absence of oxygen.

The following examples are presented for purposes of illustration, and are not intended to impose limitations on the scope of this invention.

In the Examples below, the Gardner color values were determined instrumentally, using a Color Quest XE spectrophotometer (HunterLab).

### EXAMPLE 1

### Synthesis of N,N′-di-sec-butyl-4,4′-methylenebis(benzeneamine)

4,4′-Methylenebis(benzeneamine) (9.9 g, 0.05 mol), methyl ethyl ketone (50.0 g), and Pt(S)/C (0.3 g) were charged into reactor. The reactor was purged 3 times with 84 psig of H₂ at 22° C. The reaction mixture was then stirred at 136° C. under 84 psig of H₂ for 3.5 hours. Gas chromatography (GC) showed 100% conversion of 4,4′-methylenebis(benzeneamine), and a 96% yield of N,N′-di-sec-butyl-4,4′-methylenebis(benzeneamine) as a solution in methyl ethyl ketone. The solution was almost water-white, but turned pink-red after filtration in the presence of air.

### Treatments of N,N′-di-sec-butyl-4,4′-methylenebis(benzeneamine)

A portion of the pink-red product solution was purged with nitrogen at 80° to 130° C.; the resultant neat liquid product was orange.

To another portion of the pink-red solution (2.0 g), N,N-diethylhydroxylamine (0.05 g) was added. After 5 minutes, the pink-red solution had become pale yellow.

### EXAMPLE 2

4,4′-Methylenebis(benzeneamine) (9.9 g, 0.05 mol), methyl ethyl ketone (50.0 g), Pt(S)/C (0.2 g), and N,N-diethylhydroxylamine (DEHA, 0.2 g) were charged into a reactor. The reactor was purged 3 times with 84 psig of H₂ at 22° C. The reaction mixture was then stirred at 136° C. under 84 psig of H₂ for 4 hours, after which the mixture was allowed to cool to room temperature under H₂. GC of the cooled mixture showed 80% hydrogenation, so more methyl ethyl ketone (5.0 g) was added, and the reaction was heated to 136° C. Hydrogenation seemed to be slow based on lack of uptake of H₂, so the reaction mixture was heated to 146° C. for an hour, with 115 psig of H₂; during the hour the H₂ pressure dropped to 85 psig. The temperature was raised to 150° C., and the H₂ pressure was again increased to 115 psig; the mixture was stirred under these conditions for 1.5 hours. The mixture then was allowed to cool to room temperature under H₂. GC showed a yield of 87%, so more Pt(S)/C (0.10 g) was added to the reaction mixture, which was then stirred under H₂ at 136° C. for 2 hours. The product solution was allowed to cool to room temperature. GC showed a 96% yield of N,N′-di-sec-butyl-4,4′-methylenebis(benzeneamine). The Pt(S)/C was allowed to settle out of solution (˜15-20 minutes); the solution was filtered in the presence of air to remove the remaining Pt(S)/C particles. The product solution was orange after the filtration in the presence of air.

### EXAMPLE 3

4,4′-Methylenebis(benzeneamine) (9.9 g, 0.05 mol), methyl ethyl ketone (50.0 g), Pt(S)/C (0.3 g) were charged into a reactor. The reactor was purged 3 times with 84 psig of H₂ at 22° C. The reaction mixture was then stirred at 125° C. under 84 psig of H₂ for 3.5 hours. When the product mixture was opened under a nitrogen atmosphere in a dry box, an almost water-white (very pale yellow) product solution was observed after the Pt(S)/C had settled. GC showed a 95% yield of N,N′-di-sec-butyl-4,4′-methylenebis(benzeneamine).

A vacuum (˜1-5 millitorr) was applied to the product solution to remove the methyl ethyl ketone at 22° to 25° C. After the removal of the methyl ethyl ketone was complete, the N,N′-di-sec-butyl-4,4′-methylenebis(benzeneamine) was heated to 100° C. to remove trace water. The flask containing the neat product was opened in a dry box under a nitrogen atmosphere, and N,N′-diethylhydroxylamine (˜3000 ppm, relative to N,N′-di-sec-butyl-4,4′-methylenebis(benzeneamine)) was added to the product.; the pale yellow liquid had a Gardner color of 4.5.

### EXAMPLE 4

A solution of N,N′-di-sec-butyl-4,4′-methylenebis(benzeneamine) (13.5 g; 39%; made in a manner similar to that described in the synthesis portion of Example 1) in methyl ethyl ketone was prepared in the presence of air; the solution was orange-brown in color. The methyl ethyl ketone was removed under vacuum (˜1-5 millitorr) at 25° to 30° C. The remaining liquid was then heated at 45° to 75° C. for one hour while maintaining the vacuum, and then was heated at 95° to 120° C. for one hour, still under vacuum. The brown color slowly disappeared during the 95° to 120° C. heating step. The liquid was then heated at 120° to 130° C. for two hours still under vacuum, after which the solution was cooled to room temperature under nitrogen in a dry box. The final, cooled liquid was orange in color.

### EXAMPLE 5

An orange solution of N,N′-di-sec-butyl-4,4′-methylenebis(benzeneamine) in methyl ethyl ketone was produced in a manner similar to the synthesis described in Example 1. A vacuum (˜1-5 millitorr) was applied to remove the methyl ethyl ketone; then, while maintaining the vacuum, the N,N′-di-sec-butyl-4,4′-methylenebis(benzeneamine) was heated at 40-70° C. for ˜4 hours. The final liquid was pale yellow. GC of the pale yellow liquid did not show any peaks for methyl ethyl ketone.

### EXAMPLE 6

N,N′-Di-sec-butyl-4,4′-methylenebis(benzeneamine) produced in a manner similar to the synthesis described in Example 1 was used to prepare a 27% solution in methyl ethyl ketone. Dibenzylhydroxylamine (˜2500 ppm) was added to the solution. A vacuum (˜1-5 millitorr) was applied at 25° to 38° C. to remove the methyl ethyl ketone, and then the liquid was heated to 100° C. for 1 hour while maintaining the vacuum. A pale yellow oil with a Gardner color of 4.4 was obtained.

### EXAMPLE 7

Commercially-available N,N′-di-sec-butyl-4,4′-methylenebis(benzeneamine) was mixed with N,N-diethylhydroxylamine, an optical brightener, or both N,N-diethylhydroxylamine and an optical brightener. The optical brightener was a mixture of Solvent Violet 13 (CAS # 81-48-1) and Solvent Green 3 (CAS # 128-80-3), which was used as a 0.05 wt % solution of Exalite® Blue 78-13 in a polycaprolactone polyol (Tone™ 32B8, Dow Chemical Co.). The Gardner color of the N,N′-di-sec-butyl-4,4′-methylenebis(benzeneamine) was measured before and after the mixing with the additives. The amount of each additive relative to N,N′-di-sec-butyl-4,4′-methylenebis(benzeneamine) is listed in Table 1. Results are summarized in Table 1.

TABLE 1

| Run | Initial Gardner color | Et₂NOH | Blue dye | Final Gardner color |
|---|---|---|---|---|
| 1 | 7.0 | 2 wt % | 0 | 5.5 |
| 2 | 7.0 | 0 | 9.2 ppm | 4.6 |
| 3 | 7.0 | 2 wt % | 4.6 ppm | 3.5 |
| 4 | 7.0 | 2 wt % | 9.2 ppm | 2.5 |

It is to be understood that the reactants and components referred to by chemical name or formula anywhere in this document, whether referred to in the singular or plural, are identified as they exist prior to coming into contact with another substance referred to by chemical name or chemical type (e.g., another reactant, a solvent, or etc.). It matters not what preliminary chemical changes, transformations and/or reactions, if any, take place in the resulting mixture or solution or reaction medium as such changes, transformations and/or reactions are the natural result of bringing the specified reactants and/or components together under the conditions called for pursuant to this disclosure. Thus the reactants and components are identified as ingredients to be brought together in connection with performing a desired chemical operation or reaction or in forming a mixture to be used in conducting a desired operation or reaction. Also, even though an embodiment may refer to substances, components and/or ingredients in the present tense (“is comprised of”, “comprises”, “is”, etc.), the reference is to the substance, component or ingredient as it existed at the time just before it was first contacted, blended or mixed with one or more other substances, components and/or ingredients in accordance with the present disclosure.

Also, even though the claims may refer to substances in the present tense (e.g., “comprises”, “is”, etc.), the reference is to the substance as it exists at the time just before it is first contacted, blended or mixed with one or more other substances in accordance with the present disclosure.

Except as may be expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise.

Each and every patent or other publication or published document referred to in any portion of this specification is incorporated in toto into this disclosure by reference, as if fully set forth herein.

This invention is susceptible to considerable variation within the spirit and scope of the appended claims.

1. A composition which comprises at least one aromatic secondary diamine having a Gardner color number no more than about 6, wherein said aromatic secondary diamine either is in the form of one benzene ring having two secondary amino groups on the ring, or is in the form of two benzene rings connected by an alkylene bridge and having one secondary amino group on each ring, in which composition at least one N,N-dihydrocarbylhydroxylamine is present.

2. A composition as in claim 1 wherein said aromatic secondary diamine is either:
A) in the form of one benzene ring having two secondary amino groups on the ring, which amino groups are meta or para relative to each other, or
B) in the form of two benzene rings connected by an alkylene bridge and having one secondary amino group on each ring, wherein said alkylene bridge has from one to about three carbon atoms, and wherein each amino group is meta or para relative to the alkylene bridge.

3. A composition as in claim 2 wherein said aromatic secondary diamine has one of the following characteristics:
i) one position ortho to an amino group bears a hydrocarbyl group, or
ii) each position ortho to an amino group bears a hydrocarbyl group.

4. A composition as in claim 1 wherein said N,N-dihydrocarbylhydroxylamine is present in an amount in the range of about 0.01 wt % to about 1.5 wt % based on the amount of said aromatic secondary diamine.

5. A composition as in claim 1 wherein said N,N-dihydrocarbylhydroxylamine is at least one N,N-dialkylhydroxylamine, at least one N,N′-diaraalkylhydroxylamine, or a mixture thereof.

6. A composition as in claim 1 wherein said N,N-dihydrocarbylhydroxylamine is selected from the group consisting of N,N-diethylhydroxylamine, N,N-dibenzylhydroxylamine, and a mixture thereof.

7. A composition as in claim 6 wherein said N,N-dihydrocarbylhydroxylamine is present in an amount in the range of about 0.01 wt % to about 1.5 wt % based on the amount of said aromatic secondary diamine, and wherein said aromatic secondary diamine is selected from the group consisting of N,N′-diisopropyl-2,4-diethyl-6-methyl-1,3-benzenediamine, N,N′-diisopropyl-4,6-diethyl-2-methyl-1,3-benzenediamine, or a mixture thereof; N,N′-di-sec-butyl-2,4-diethyl-6-methyl-1,3-benzenediamine, N,N′-di-sec-butyl-4,6-diethyl-2-methyl-1,3-benzenediamine, or a mixture thereof; N,N′-diisopropyl-4,4′-methylenebis(2,6-diethylbenzeneamine); N,N′-di-sec-butyl-4,4′-methylenebis(2,6-diethylbenzeneamine), and N,N′-di-sec-butyl-4,4′-methylenebis(benzeneamine).

8. A composition as in claim 1 which further comprises at least one optical brightener.

9. A composition as in claim 8 wherein said optical brightener is present in an amount in the range of about 1 ppm to about 10 ppm relative to the aromatic secondary diamine.

10. A process for reducing color in an aromatic secondary diamine, which process comprises heating, while under a vacuum, at least one aromatic secondary diamine, in the presence of at least one N,N-dihydrocarbylhydroxylamine, wherein said aromatic secondary diamine either is in the form of one benzene ring having two secondary amino groups on the ring, or is in the form of two benzene rings connected by an alkylene bridge and having one secondary amino group on each ring.

11. A process as in claim 10 wherein said aromatic secondary diamine is either:
A) in the form of one benzene ring having two secondary amino groups on the ring, which amino groups are meta or para relative to each other, or
B) in the form of two benzene rings connected by an alkylene bridge and having one secondary amino group on each ring, wherein said alkylene bridge has from one to about three carbon atoms, and wherein each amino group is meta or para relative to the alkylene bridge.

12. A process as in claim 11 wherein said aromatic secondary diamine has one of the following characteristics:
i) one position ortho to an amino group bears a hydrocarbyl group, or
ii) each position ortho to an amino group bears a hydrocarbyl group.

13. A process as in claim 10 wherein a N,N-dihydrocarbylhydroxylamine is present during said process, and wherein said N,N-dihydrocarbylhydroxylamine is present in an amount in the range of about 0.5 wt % to about 5 wt % based on the amount of said aromatic secondary diamine.

14. A process as in claim 10 wherein a N,N-dihydrocarbylhydroxylamine is present during said process, and wherein said N,N-dihydrocarbylhydroxylamine is at least one N,N-dialkylhydroxylamine, at least one N,N-diaraalkylhydroxylamine, or a mixture thereof.

15. A process as in claim 14 wherein said N,N-dihydrocarbylhydroxylamine is selected from the group consisting of N,N-diethylhydroxylamine, N,N-dibenzylhydroxylamine, and a mixture thereof.

16. A process as in claim 15 wherein said N,N-dihydrocarbylhydroxylamine is present in an amount in the range of about 0.5 wt % to about 5 wt % based on the amount of said aromatic secondary diamine, and wherein said aromatic secondary diamine is selected from the group consisting of N,N′-diisopropyl-2,4-diethyl-6-methyl-1,3-benzenediamine, N,N′-diisopropyl-4,6-diethyl-2-methyl-1,3-benzenediamine, or a mixture thereof; N,N′-di-sec-butyl-2,4-diethyl-6-methyl-1,3-benzenediamine, N,N′-di-sec-butyl-4,6-diethyl-2-methyl-1,3-benzenediamine, or a mixture thereof; N,N′-diisopropyl-4,4′-methylenebis(2,6-diethylbenzeneamine); N,N′-di-sec-butyl-4,4′-methylenebis(2,6-diethylbenzeneamine), and N,N′-di-sec-butyl-4,4′-methylenebis(benzeneamine).

17. A process as in claim 10 wherein an aromatic secondary diamine having a Gardner color number less than about 6 is obtained.

18. A process for forming an aromatic secondary diamine which comprises mixing together a ketone or aldehyde and an aromatic primary diamine in the presence of a hydrogenation agent, wherein said aromatic primary diamine is either is in the form of one benzene ring having two primary amino groups on the ring, or is in the form of two benzene rings connected by an alkylene bridge and having one primary amino group on each ring, characterized in that the process is conducted in the substantial absence of oxygen, and in the presence of at least one N,N-dihydrocarbylhydroxylamine, such that an aromatic secondary diamine either in the form of one benzene ring having two secondary amino groups on the ring, or in the form of two benzene rings connected by an alkylene bridge and having one secondary amino group on each ring is formed.

19. A process as in claim 18 wherein said aromatic primary diamine is either:
A) in the form of one benzene ring having two primary amino groups on the ring, which amino groups are meta or para relative to each other, or
B) in the form of two benzene rings connected by an alkylene bridge and having one primary amino group on each ring, wherein said alkylene bridge has from one to about three carbon atoms, and wherein each amino group is meta or para relative to the alkylene bridge.

20. A process as in claim 18 wherein said aromatic primary diamine has one of the following characteristics:
i) at least one position ortho to an amino group bears a hydrocarbyl group, or
ii) each position ortho to an amino group bears a hydrocarbyl group.

21. A process as in claim 18 wherein said ketone or aldehyde has at least one of the following characteristics:
a) from about three to about fifteen carbon atoms;
b) the hydrocarbyl portion of said ketone or aldehyde is an aliphatic straight chain or a branched aliphatic group.

22. A process as in claim 18 wherein said process is conducted in the presence of hydrogen and a hydrogenation catalyst, wherein said hydrogenation catalyst is sulfided platinum on carbon, sulfided palladium on carbon, or a mixture thereof.

23. A process as in claim 18 wherein said N,N-dihydrocarbylhydroxylamine is at least one N,N-dialkylhydroxylamine, at least one N,N-diaraalkylhydroxylamine, or a mixture thereof.

24. A process as in claim 18 wherein said N,N-dihydrocarbylhydroxylamine is present in an amount in the range of about 0.5 wt % to about 5 wt % based on the amount of said aromatic primary diamine.

25. A process as in claim 18 which further comprises heating, while under a vacuum, at least a portion of said aromatic secondary diamine, optionally in the presence of at least one N,N′-dihydrocarbylhydroxylamine.

26. A process as in claim 25 wherein said N,N-dihydrocarbylhydroxylamine is at least one N,N-dialkylhydroxylamine, at least one N,N-diaraalkylhydroxylamine, or a mixture thereof.

27. A process as in claim 26 wherein said N,N-dihydrocarbylhydroxylamine is selected from the group consisting of N,N-diethylhydroxylamine, N,N-dibenzylhydroxylamine, and a mixture thereof.

28. A process as in claim 25 wherein a N,N-dihydrocarbylhydroxylamine is present during said process, and wherein said N,N-dihydrocarbylhydroxylamine is present in an amount in the range of about 0.5 wt % to about 5 wt % based on the amount of said aromatic secondary diamine.

29. A process as in claim 27 wherein said N,N-dihydrocarbylhydroxylamine is present in an amount in the range of about 0.5 wt % to about 5 wt % based on the amount of said aromatic secondary diamine, wherein said aromatic primary diamine is selected from the group consisting of 1,3-benzenediamine; 1,4-benzenediamine; 2,4-diethyl-6-methyl-1,3-benzenediamine, 4,6-diethyl-2-methyl-1,3-benzenediamine, and mixtures thereof; 4,4′-methylenebis(2,6-diethylbenzeneamine); and 4,4′-methylenebis(benzeneamine), and wherein said ketone or aldehyde is a ketone, and is acetone, methyl ethyl ketone, 4-methyl-2-pentanone, 3,3-dimethyl-2-butanone, cyclohexanone, 4-heptanone, or 5-nonanone.

30. A process as in claim 25 wherein an aromatic secondary diamine having a Gardner color number less than about 6 is obtained.

31. A process as in claim 29 wherein an aromatic secondary diamine having a Gardner color number less than about 6 is obtained.
